(19) United States
(12) Patent Application Publication
(10) Pub. No.: US 2008/0206360 A1
(43) Pub. Date: Aug. 28, 2008

(54) Folate based migraine treatment

(76) Inventor: Curt Hendrix, Encino, CA (US)

Correspondence Address: VENABLE LLP, P.O. BOX 34385, WASHINGTON, DC, 20043-9998, US

(21) Appl. No.: 12/068,110

(22) Filed: Feb. 1, 2008

Related U.S. Application Data
(63) Continuation of application No. 11/002,750, filed on Dec. 1, 2004, now abandoned.

(51) Int. Cl. A61K 33/06; A61P 29/00

(52) U.S. Cl. 424/682

(57) ABSTRACT

Formulations for the prevention and treatment of headaches, particularly migraine headaches, comprise folate in combination with one or more of riboflavin, feverfew, or an extract of feverfew, magnesium in the form of acid salts, magnesium oxide, complexes or chelates and/or other nutritional supplements such as vitamin B6, B12, betaine, and plant derived phytochemicals or synthesized versions thereof.

This application is directed to new formulations for the prevention and treatment of headaches, particularly migraine headaches, which comprise folate in combination with compounds known to be beneficial in migraine treatment, such as riboflavin (vitamin B2), feverfew, or an extract of feverfew, magnesium in the form of acid salts, magnesium oxide, complexes or chelates and /or other nutritional supplements such as vitamin B6, B12, betaine, and plant derived phytochemicals or synthesized versions thereof.

### BACKGROUND

Folic acid or salts thereof, referred to as folates, along with vitamins B₆ and B₁₂ are required in metabolic pathways involving methionine, homocysteine, cystathionine, and cysteine. The term folates as used herein is meant to include, as a minimum, folacin (USP folic acid), naturally occurring folinic acid, 5-methyl tetrahydrofolate, and tetra hydrofolate as well as salts or metabolites of these compounds. It appears that all three compounds (Folate, B₆ and B₁₂) are necessary for normal metabolism. However, these three compounds each function in a different manner. Folate, even if available at normal levels, is consumed in the metabolic process and therefore must be constantly replenished by diet or supplements. However, B6 and B12 function as co-factors. While necessary for the metabolic process to proceed, they are each regenerated in the process. Therefore, if they are present in normal amounts in serum, supplementation may not be necessary. B₁₂ in the form of 5′-deoxyadenosylcobalamin is an essential cofactor in the enzymatic conversion of methylmalonylCoA to succinylCoA. The remethylation of homocysteine (HC) to methionine catalyzed by methionine synthase requires folate (methyltetrahydrofolate) and B₁₂ in the form of methylcobalamin. HC is condensed with serine to form cystathionine (CT) in a reaction catalyzed by cystathionine beta.-synthase which requires B₆ (pyridoxal phosphate). CT is also hydrolyzed in another B₆-dependent reaction to cysteine and alpha.-ketobutyrate. Homocysteine is a modified form of the amino acid methionine that is tightly regulated by enzymes which require folate. By impairing DNA repair mechanisms and inducing oxidative stress, homocysteine can cause the dysfunction or death of cells in the cardiovascular and nervous systems. Homocysteine appears to be present in many disease states. However, dietary folate stimulates homocysteine removal and may thereby protect cells against disease processes.

The principal biochemical function of folates is the mediation of one-carbon transfer reactions. 5-Methyltetrahydrofolate donates a methyl group to homocysteine, in the conversion of homocysteine to L-methionine. The enzyme that catalyzes the reaction is methionine synthase. Vitamin B₁₂ is a cofactor in the reaction. This reaction, in which folate and vitarnin B₁₂ are coparticipants, is of great importance in the regulation of serum homocysteine levels. The L-methione produced in the reaction can participate in protein synthesis and is also a major source for the synthesis of S-adenosyl-L-methionine (SAMe). The methyl group donated by 5-methyltetrahydrofolate to homocysteine in the formation of L-methionine is used by SAMe in a number of transmethylation reactions involving nucleic acids, phospholipids and proteins, as well as for the synthesis of epinephrine, melatonin, creatine and other molecules. Tetrahydrofolate is the folate product of the methionine synthase reaction. 5-Methyltetrahydrofolate is generated in by conversion of 5,10-methylenetetrahydrofolate into 5-methyltetrahydrofolate via the enzyme methyleneterahydrofolate reductase (MTHFR). 5,10-Methylenetetrahydrofolate is regenerated from tetrahydrofolate via the enzyme serine hydroxymethyltransferase, a reaction, which in addition to producing 5,10-methylenetetrahydrofolate, yields glycine.

5,10-Methylenetetrahydrofolate, in addition to its role in the metabolism of homocysteine, supplies the one-carbon group for the methylation of deoxyuridylic acid to form the DNA precursor thymidylic acid. This reaction is catalyzed by thymidylate synthase and the folate product of the reaction is dihydrofolate. Dihydrofolate is converted to tetrahydrofolate via the enzyme dihydrofolate reductase.

Folates are also involved in reactions leading to de novo purine nucleotide synthesis, interconversion of serine and glycine, generation and utilization of formate, the metabolism of L-histidine to L-glutamic acid, the metabolism of dimethylglycine to sarcosine and the metabolism of sarcosine to glycine.

One of the natural folates, folinic acid, is used as a pharmaceutical agent. Folinic acid, also known as leucovorin, citrovorum factor and 5-formyltetrahydrofolate, is used as rescue therapy following high-dose methotrexate in the treatment of osteosarcoma. It is also used to diminish the toxicity of methotrexate. It is used in the treatment of megaloblastic anemia due to folate deficiency and in the prevention or treatment of the toxic side effects of trimetrexate and pyrimethamine. The combination of folinic acid and 5-fluorouracil has until recently been standard therapy for metastatic colorectal cancer. Folinic acid increases the affinity of flurouracil for thymidylate synthase. Folinic acid is available as a calcium salt for parenteral or oral administration.

In addition to being known as pteroylglutamic acid or PGA, folic acid is known chemically as N-[4-[[(2-amino-1,4-di-hydro-4-oxo-6-pteridinyl)methyl]amino]benzoyl]-L-glutamic acid. Older names for folic acid are vitamin B₉, folicin, vitamin Bc and vitamin M. Its molecular formula is C₁₉H₁₉N₇O₆ and its molecular weight is 441.40 daltons. Folic acid forms yellowish-orange crystals. The color is imparted by the pteridine ring of folic acid. Pteridine also imparts color to butterfly wings.

Folate has been prescribed as a nutritional supplement for many medical conditions based on the presence of elevated homocysteine levels which occur in those conditions. Folate supplements appear to reverse the elevated homocysteine levels. However, the elevated homocysteine level may be a result of inadequate supply or excessive consumption of folate and not the cause of the disease. It is clinically beneficial in such instances to provide folate supplements as individuals with elevated homocysteine levels appear to be at increased risk for cardiovascular disease and stroke, and neurodegenerative disorders such as Alzheimer's and Parkinson's diseases as well as neural tube defects, spontaneous abortion, placental abruption, low birth weight, renal failure, rheumatoid arthritis, alcoholism, osteoporosis, neuropsychiatric disorders, non-insulin-dependent diabetes and complications of diabetes, fibromyalgia and chronic fatigue syndrome. Moderate elevations of HC might be associated with increased risk for vascular disease (Ueland et al. (1992) in Atherosclerotic Cardiovascular Disease, Hemostasis, and Endothelial Function (Francis, Jr., ed.), Marcel Dekker, Inc., New York, pp. 183-23). However, folic acid deficiencies have also been associated with periphereal vascular disease and coronary disease in individuals with normal homocysteine levels (Bunout, D. et al “low Serum Folate but Normal Homocysteine Levels in Patients with Atheroslerotic Vascular Disease and Matched Healthy Controls”, Nutrition 2000, 16, p 434-8) suggesting that folates may have a protective effect that extends beyond maintaining normal homocysteine levels. In addition, moderate hyperhomocysteinaemia has been shown to be frequently present in cases of stroke and to be independent of other stroke risk factors (Brattstrom et al. (1992) Eur. J. Clin. Invest. 22:214-221).

It is not clear if the various disease states are caused by elevated homocysteine levels or the elevated homocysteine levels are caused by other factors which are the primary cause of the disease state and result in elevated levels of homocysteine. For example, it is also known that folate supplements are usefully where B₁₂ deficiencies exist, but homocysteine levels may not be elevated. Individuals with B₁₂ deficiency can display neurologic disorders, typically relating to underlying anemia. However, supplementing diet with only folate is not medically recommended as these folate supplements may mask the underlying B₁₂ problem. U.S. Pat. No. 4,945,083, issued Jul. 31, 1990 to Jansen, entitled Safe Oral Folic Acid-Containing Vitamin Preparation, describes an oral vitamin preparation comprising the combination of 0.1-1.0 mg B₁₂ and 0.1-1.0 mg folate for the treatment or prevention of megaloblastic anemia.

Normal serum folate levels in healthy individuals are 2.5-20 ng/ml, with levels less than 2.5 ng/ml indicating the possibility of clinically significant deficiency. Like B₁₂ serum levels, however, serum folate levels are a relatively insensitive measure in that only 50-75% of patients with folate deficiency have levels less than 2.5% ng/ml, with most of the remaining 25-50% being in the 2.5-5.0 ng/ml range (Allen (1991), Cecil Textbook of Medicine, 19th Ed.).

A series of patents to Allen et al, (U.S. Pat. No. 5,563,126, U.S. Pat. No. 5,795,873, U.S. Pat. No. 6,207,651, U.S. Pat. No. 6,297,224 and U.S. Pat. No. 6,528,496)) teaches the use of oral compositions or a transdermal patch delivering a combination of B₁₂ and folate, or B₁₂, folate and B6, in concentrations sufficient to reduce elevated homocysteine levels by treating either single or multiple deficiencies of B₁₂, folate, and B₆. The Allen non-prescription formulations include 0.3-10 mg CN-cobalamin (B₁₂) and 0.1-0.4 mg folate or 0.3-10 mg B₁₂, 0.1-0.4 folate, and 5-75 mg B₆. The Allen prescription formulations comprise between 0.3-10 mg CN-cobalamin (B₁₂) and 0.4-10.0 mg folate or 0.3-10 mg B₁₂, 0.4-1.0 mg folate, and 5-75 mg B₆.

### DESCRIPTION OF INVENTION

Applicant has discovered that dietary supplementation with folate may be beneficial in treating certain medical conditions. In particular, compositions set forth herein, which include folates, have been found to be beneficial in preventing, reducing the severity of, or decreasing the incidence of various forms of headache, including but not limited to migraine headaches, chronic daily headaches and tension headaches, even though the individual does not appear to have a B₁₂ deficiency or elevated homocysteine levels. These compositions may also be beneficial in preventing B₁₂ deficiencies or elevated homocysteine levels. Homocysteine levels have been found to not be elevated in migraine suffers (Hering-Hanit, R. et al, “Is Blood Homocysteine Elevated in Migraine?”, Headache: The Journal of Head and Face Pain, 41, 8, pg 779, September 2001) and the reduction of homocysteine levels, whether normal or elevated, has not been shown to reduce the incidence or severity of migraines. Lea et al (Lea, R. A. et al, “The. Methylenetetrahydrofolate Reductase Gene Variant C677T Influences Susceptibility to Migraine”, BMC Medicine 2004, 2, 3, http//www.biomedcentral. com/1741-7015/2/3) discusses the C677T genetic variant and its possible association with elevated levels of homocysteine and homocysteine-related endothelial dysfunction. He recognizes that migraine pathophysiology is partly explained by changes in vascular tension and altered cerebral blood flow and concludes that migraine with aura may be related to a rare genetic inability to process folate. If elevated homocysteine occurs, it is not a cause of migraine but a result of an inadequate supply of folate. Applicant has found that increasing serum folate (i.e. by delivery of folates) is beneficial in reducing the incidence or severity of migraine headaches irrespective of whether homocysteine is normal or elevated. In other words, increasing folate levels in the body is beneficial in treating headaches, particularly migraine. These increased levels may reduce homocysteine levels or prevent elevated homocysteine levels but homocysteine levels are unrelated to the occurrence or severity of the headaches.

It is also known that providing magnesium to a migraine sufferer or increasing adsorption of magnesiurn may be beneficial in stemming migraine (Hendrix, C. U.S. Pat. Nos. 6,068,999 and 6,500,450, incorporated herein by reference) and that certain B vitamins are necessary for proper utilization of magnesium. Some people with fibromyalgia or chronic fatigue syndrome, which appear to be responsive to increased magnesium, appear to also have B vitamin deficiencies, especially B₁₂ deficiencies. One study (Regland, B et al, “Increased Concentrations of Homocysteine in the Cerebrospinal Fluid in Patients with Fibromyalgia and Chronic Fatigue Syndrome”, Scand J Rheumatol.;26(4): (1997) 301-7) has shown that homocysteine levels are high in the cerebrospinal fluid of fibromyalgia patients, and this indicates low levels of B₁₂ in the brain. Additionally, homocysteine was believed to cause a depletion of intracellular free magnesium (Li, W. et al .“Extracellular Magnesium Regulates Effects Of Vitamin B6, B₁₂ And Folate On Homocysteinemia-Induced Depletion Of Intracellular Free Magnesium Ions In Canine Cerebral Vascular Smooth Muscle Cells: Possible Relationship To [Ca2+]I, Atherogenesis And Stroke”, Neurosci Lett., 274,2 (1999) p 83-6) and only a combination of B₆, B₁₂, and folate acid would stop the depletion of magnesium. However, it is again not clear if homocysteine is a cause of the magnesium depletion or an effect of the folate, B₆ and B12 deficiencies.

S-adenosylmethionine (SAMe) is a substance that occurs naturally in the body. It is the combination of one (1) essential amino acid and ATP that plays a role in 35-40 biochemical reactions throughout the body. In most people, the body can make all the SAMe it needs, but some patients with depression and other conditions have been found to have lower levels of the compound as well as lower levels of folate and vitamin B₁₂. These three substances each play a part in the metabolic process of “methyl donation” or “methylation”, a process in which a molecule comprised of one (1) carbon molecule and three (3) hydrogen atoms is attached to proteins and lipids. These methylation reactions are involved in the production of the neurotransmitters serotonin and dopamine in the brain and enzymes that help repair joints and the liver. There is evidence that serotonin is a factor in migraine and is involved in the so called “rebound effect”, because of its vasoconstricting effect when serotonin levels are elevated and subsequent vasodilation as serotonin levels decrease. Coincidently, folate deficiency also appears to reduce brain serotonin and contribute to depression in individuals. By supplementing the diet with folate, serotonin generation and its metabolism is balanced, depression decreases and the cycling of vasodilation and vasoconstriction caused by fluctuation in serotonin is minimized.

Use of folate compositions as set forth herein are effective for treating headaches, particularly migraines, because of its ability to improve endothelial function and retard blood vessel dilation and constriction (one of the common conditions present during a migraine episode). Additionally folate supplementation is beneficial in normalizing serum levels of several biochemicals which, if not controlled, influence the incidence, severity or extent of migraine, including but not limited to B vitamins, particularly. B₂, B₆ and B₁₂, magnesium absorption, and controlled generation and metabolism of serotonin. While folic acid may typically be prescribed to patients as daily diet supplements or in prescription dosages to reduce homocysteine levels, as indicated above, studies have shown that there is no correlation between homocysteine levels and migraine headaches and in fact homocysteine levels are typically normal in individuals suffering from migraines, in the absence of other diseases which may be related to hyperhomocystemia.

While it is known that the delivery of certain minerals, vitamins and natural products or their extracts, particularly feverfew, or parthenolide extracted from feverfew, magnesium and riboflavin, are beneficial in preventing or reducing the incidence or severity of migraine headaches, it has now been found that delivery of folates alone or, in combination with one or more of these compounds previously known to be beneficial in treating migraines, or other compositions effective in addressing migraine symptoms, provides further therapeutically benefit in treating headache, particularly migraine. In particular, folate compositions have now been found to address inflammation, for exanple caused by NO, as well as endothelial function. Nitrogen oxide (NO) synthase creates NO which is inflammatory to tissue and as a result can cause or worsen a migraine. The beneficial properties of folates can also be enhanced by the concurrent use of certain B vitamins, particularly pyridoxal-5-phosphate (P5P) and hydrocobalamin, antioxidants, such as vitamin E, SAMe and CoQ10. Addition of NO synthase inhibitors, such as amino guanidine, L-carnitine, asymmetric argentine, and certain plant derived phytochemicals can enhance the inflammation reducing properties of folates.

In particular, it has been discovered that folate in daily dosages in excess of 100 mcg, particularly in concentrations of 100 mcg to about 10 mg. in combination with additional compounds found to be effective in treating migraine provides a more effective and reliable composition when taken daily for preventing the occurrence of migraine. Further, should migraine headaches occur, with or without the preventive dosages, the migraine symptoms can be reduced in severity or eliminated and the recurrence of the migraine, or the rebound effect, can be eliminated or reduced by folate compositions, which may include elevated concentrations of folate as well as on or more of the additional compounds described herein.

Compounds which have been found to be effective in combination with folates for these purposes include, but are not limited to, magnesium in the form of oxides, complexes, chelates or acid salts (i.e. magnesium citrate, magnesium acetate, magnesium tartrate, etc.), calcium, zinc, feverfew and extracts from feverfew (parthenolide alone or in combination with other feverfew extractables), , essential fatty acids, various vitamins such as niacin, niacinamide, riboflavin (vitamin B2), the various forrns of vitamin B-6 (especially pyridoxal-5-phosphate (P-5-P)), the various forms of vitamin B12 (cyanocobalamin, methylcobalamin, hydroxycobalamin, etc.) and Vitamin C and metabolites such as dehydroascorbic acid, triptan drugs, ergotamines, CoQ-10, 5 HTP, mast cell stabilizers, various analgesic or pain relievers such as aspirin, ibuprofen, and acetaminophen, channel blockers, beta blockers, i-NOs inhibitors (inducible nitric oxide inhibitors), NMDA antagonists (for example ketarnine, memantine, DL-2-aminophosphonovaleric acid (APV) and NAC) melatonin, taurine, homo-taurine, lysine, GABA and the nicotinate form of GABA (GABA(nictinoyl)), epilepsy drugs such as Valproate, compounds which increase GABA levels such as topiramate, glycine, progesterone, DLPA (D,L-phenylalanine) D-phenylal anine and L-phenylalanine, quercetin, histamine, isoflavones, curcumin and metabolites and derivatives thereof, aminoguanidine, L-carnosine, indoles, isothiocyanates and derivatives thereof, corydalis (dl-tetrahydropalmatine), and various natural products, such as ginger, butterbur, nettle leaf, devils claw, chaste berry, yellow jasmine, black cohash, dong quai, piscidia erythrina (jamaican dogwood), viburnum and extracts and phytrochemicals derived therefrom While particular compounds or forms of a compound are listed above it should be recognized that it is contemplated that each of these materials can be provided as metabolites, derivatives, salts or other active forms of the listed material.

Particularly preferred compositions are:

Formula 1—Daily doses of about 100 mcg., preferably 100 mcg. to 10 mg, of folate along with one or more of
compounds containing at least about 100 mg, preferably 100 mg. to 1,000 mg magnesium, for example as an oxide, salt, chelate, etc.,
at least about 100 mcg of vitamin B12, preferably in the hydroxycobalamine
form, preferably 100 mcg. to about 2 mg.,
at least about 1 mg B6 (pyridoxal-5-phosphate or pyridoxamine) preferably about 1 mg to about 100 mg,
at least about 25 mg riboflavin (B2), preferably 25-1,000 mg, and
at least about 100 mg. vitamin C (ascorbic acid or dehydroascorbic acid),
preferably about 100 mg.-3 g.

A more preferred composition contains:
at least 1mg folate , preferably 5 mg folate,
360-400 mg of available magnesium,
about 400 mg of vitamin B2,
about 50 mg vitamin B6, preferably in the form of P-5-P,
about 1 mg vitamin B12, and
about 250 mg of vitamin C).

Formula 2—Same as Formula 1 with at least about 50 mg., preferably 50 mg to 500 mg. of CoQ-10 substituted for the riboflavin.

Formula 3—For Acute treatment—Folate, vitamins B12, B6, B2 and C and magnesium in the same amounts as set forth in Formula 1, with the addition of one or more of piscidia erythrina Oamaican dogwood), corydalis (dl-tetrahydropalmatine), DLPA (D,L-phenylalanine), viburnum, butterbur or feverfew or an extract of feverfew (parthenolide).

While specific formulations or combinations of compounds have been set forth as beneficial or preferred, the invention is not limited to those combinations, or compositions listed herein as other compounds, whether natural, or synthesized may be discovered to be active in treating or preventing migraines. Also, many of the compounds, particularly vitamins and minerals are essential to normal diet and are not toxic or are not toxic unless provided in very high quantities. Therefore, while minimum levels are desired, one skilled in the art will recognize that upper limits may be varied without being outside the scope of the invention. The invention is limited only by the claims set forth herein which include folate in combination with other active ingredients for prevention or treatment of headaches, particularly migraine headaches.

It is preferred that these compositions be delivered orally and the components be prepared for ingestion by the headache sufferer in a manner that makes the composition available in therapeutically effective amounts. As such, they may be prepared as water soluble compositions, delivered in liquid form, lyophilized, encapsulated, or in a manner suitable for time release, delayed release or enteric delivery, or any manner typically used for orally delivered pharmaceuticals, nutraceuticals or vitamins, or combined with foods or other normally ingested products. However, the invention is not limited to oral delivery as the compositions set forth herein may also be delivered by nasal spray, inhalation techniques, transdermally, transmucossal, by suppository, injected or by intravenous methods.

1. (canceled)

2. A pharmaceutical composition comprising a daily dose of
at least about 100 mcg of folate,
at least about 100 mg of available magnesium,
at least about 100 mcg of vitamin B₁₂,
at least about 1 mg of vitamin B₆,
at least about 25 mg of vitamin B₂, and
at least about 100 mg of vitamin C.

3. The pharmaceutical composition of claim 2 wherein a daily dose contains less than about 10 mg of folate, less than about 1000 mg of available magnesium, less than about 2 mg of vitamin B₁₂, less than about 100 mg of vitamin B₆, less than about 1000 mg of vitamin B₂ and less than about 3 grams of vitamin C.

4. The pharmaceutical composition of claim 3 wherein the available magnesium is in the form of an oxide, salt, or chelate, vitamin B₁₂ is in the form of hydroxycobalamin, vitamin B₆ is in the form of pyridoxal-5-phosphate or pyridoxamine, or both, and vitamin C is in the form of ascorbic acid or dehydroascorbic acid or both.

5. The pharmaceutical composition of claim 2 comprising a daily dose of:
about 1 mg to about 5 mg of folate,
about 360 to 400 mg of available magnesium,
about 400 mg of vitamin B₂,
about 50 mg of vitamin B₆,
about 1 mg of vitamin B₁₂, and
about 250 mg of vitamin C.

6. A pharmaceutical composition comprising a daily dose of:
at least about 100 mcg of folate,
compounds containing at least about 100 mg of available magnesium in the form of an oxide, salt, or chelate,
at least about 100 mcg of the hydroxycobalamin form of vitamin B₁₂,
at least about 1 mg of vitamin B₆,
at least about 50 mg of coenzyme Q-10, and
at least about 100 mg of vitamin C.

7. The pharmaceutical composition of claim 2 further comprising one or more of D or L or D,L-phenylalanine, viburnum, butterbur or feverfew or an extract of feverfew.

8. (canceled)

9. The medicinal composition of claim 7 wherein the extract from feverfew is parthenolide alone or in combination with other feverfew extractables.

10. (canceled)

11. (canceled)

12. A method of inhibiting, treating or reducing the severity or frequency of headaches comprising providing a daily dose of:
at least about 100 mcg of folate,
compounds containing at least about 100 mg of available magnesium,
at least about 100 mcg of vitamin B₁₂,
at least about 1 mg of vitamin B₆,
at least about 25 mg of vitamin B₂, and
at least about 100 mg of vitamin C.

13. The method of claim 12 wherein the daily dose contains less than about 10 mg of folate, and one or more of less than about 1000 mg available magnesium, less than about 2 mg of vitamin B₁₂, less than about 100 mg of vitamin B₆, less than about 1000 mg of vitamin B₂ and less than about 3 grams of vitamin C.

14. The method of claim 12 wherein the available magnesium is in the form of an oxide, salt, or chelate, vitamin B₁₂ is in the form of hydroxycobalamin, vitamin B₆ is in the form of pyridoxal-5-phosphate or pyridoxamine, and vitamin C is in the form of ascorbic acid or dehydroascorbic acid.

15. The method of claim 12 comprising providing a daily dose of:
about 1 mg to about 5 mg of folate,
about 360 to 400 mg of available magnesium,
about 400 mg of vitamin B₂,
about 50 mg of vitamin B₆,
about 1 mg of vitamin B₁₂, and
about 250 mg of vitamin C.

16. A method of inhibiting, treating or reducing the severity or frequency of headaches comprising providing a daily dose of 100 mcg to 10 mg of folate along with one or more of compounds containing 100 mg to 1,000 mg available magnesium in the form of an oxide, salt, or chelate,
100 mcg to 1 mg of the hydroxycobalamin form of vitamin B₁₂,
1 mg to 100 mg of vitamin B₆ in the form of pyridoxal-5-phosphate or pyridoxamine,
50 mg to 500 mg of coenzyme Q-10, and
100 mg to 3 grams of vitamin C in the form of ascorbic acid or dehydroascorbic acid.

17. The method of claim 12 further comprising providing a composition including one or more of D,L-phenylalanine, viburnum, butterbur or feverfew or an extract of feverfew.

18. (canceled)

19. The method of claim 17 wherein the extract of feverfew is parthenolide alone or in combination with other feverfew extractables.

20. (canceled)

21. The pharmaceutical composition of claim 5, wherein the vitamin B₆ is in the form of pyridoxal-5-phosphate.

22. A pharmaceutical composition comprising a daily dose of at least about 100 mcg of folate, at least about 360 mg of available magnesium, and one or more of:
at least about 100 mcg of vitamin B₁₂,
at least about 1 mg of vitamin B₆,
at least about 25 mg of vitamin B₂,
at least about 50 mg of coenzyme Q-10, and
at least about 100 mg of vitamin C.

23. The pharmaceutical composition of claim 22, wherein the daily dose contains at least about 1 mg folate.

24. The pharmaceutical composition of claim 22, comprising:
at least about 100 mcg of folate,
at least about 360 mg of available magnesium,
at least about 100 mcg of vitamin B₁₂,
at least about 1 mg of vitamin B₆,
at least about 100 mg of vitamin C, and one or more of:
at least about 25 mg of vitamin B₂, and
at least about 50 mg of coenzyme Q-10.

25. The pharmaceutical composition of claim 22, comprising a daily dose of about 1 mg to about 5 mg of folate, from about 360 to about 400 mg of available magnesium, and one or more of:
about 400 mg of vitamin B₂,
about 50 mg of vitamin B₆,
about 1 mg of vitamin B₁₂,
from about 50 mg to about 500 mg coenzyme Q-10, and
about 250 mg of vitamin C.
